(19) United States
(12) Patent Application Publication
(10) Pub. No.: US 2009/0247599 A1
(43) Pub. Date: Oct. 1, 2009

(54) METHODS OF TREATING CELL PROLIFERATIVE DISORDERS USING A COMPRESSED TEMOZOLOMIDE DOSING SCHEDULE

(75) Inventors: Walter Robert Bishop, Pompton Plains, NJ (US); Paul Kirschmeier, Basking Ridge, NJ (US); Ming Liu, Fanwood, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 12/420,109

(22) Filed: Apr. 8, 2009

Related U.S. Application Data
(63) Continuation of application No. 11/593,839, filed on Nov. 7, 2006, now abandoned.
(60) Provisional application No. 60/734,162, filed on Nov. 7, 2005.

(51) Int. Cl. A61K 31/4188; A61P 35/00

(52) U.S. Cl. 514/393

(57) ABSTRACT

There are disclosed methods and kits for treating cancer in a patient in need of such treating comprising administering temozolomide according to improved dosing schedules.

This application claims priority from U.S. Provisional Application No. 60/734,162, filed Nov. 7, 2005, the entirety of which is incorporated by reference as if set forth fully herein.

### FIELD OF THE INVENTION

This invention describes novel methods and kits for treating subjects afflicted with a proliferative disease such as cancer, a tumor, or metastatic disease.

### BACKGROUND OF THE INVENTION

Stupp et al., J. Clin. Onc., 20(5):1375-1382 (2002), report that brain tumors comprise approximately 2% of all malignant diseases. However, it is stated that with an incidence of 5 per 100,000 persons, more than 17,000 cases are diagnosed every year in the United States, with approximately 13,000 associated deaths. In adults, Stupp et al. report, the most common histologies are grade 3 anaplastic astrocytoma and grade 4 glioblastoma multiforme (“GBM”). According to Stupp et al., the standard management of malignant gliomas involves cytoreduction through surgical resection, when feasible, followed by radiotherapy (RT) with or without adjuvant chemotherapy. However, Stupp et al. report that despite this multidisciplinary approach, the prognosis for patients with GBM remains poor. The median survival rates for GBM are reported to be typically in the range of 9 to 12 months, with 2-year survival rates in the range of only 8% to 12%.

Nitrosoureas are the main chemotherapeutic agents used in the treatment of malignant brain tumors. However, they have shown only modest antitumor activity. Although frequently prescribed in the United States, the benefit of adjuvant chemotherapy with single-agent carmustine (BCNU) or lomustine or the combination regimen procarbazine, lomustine, and vincristine has never been conclusively demonstrated.

Chemotherapeutic efficacy, the ability of chemotherapy to eradicate tumor cells without causing lethal host toxicity, depends on drug selectivity. One class of anticancer drugs, alkylating agents, cause cell death by chemically modifying DNA which creates base pair mismatches and prevents DNA replication and transcription. In normal cells, the damaging action of alkylating agents can be repaired by cellular DNA repair enzymes, in particular O⁶-methylguanine-DNA methyltransferase (MGMT) also known as O⁶-alkylguanine-DNA-alkyltransferase (AGAT). The level of MGMT varies in tumor cells, even among tumors of the same type. The gene encoding MGMT is not commonly mutated or deleted. Rather, low levels of MGMT in tumor cells are due to an epigenetic modification; the promoter of the MGMT gene is methylated, thus preventing expression of MGMT.

Methylation has been shown by several lines of evidence to play a role in gene expression, cell differentiation, tumorigenesis, X-chromosome inactivation, genomic imprinting and other major biological processes. In eukaryotic cells, methylation of cytosine residues that are immediately 5′ to a guanosine, occurs predominantly in cytosine-guanine (CG) poor regions. In contrast, CpG islands remain unmethylated in normal cells, except during X-chromosome inactivation and parental specific imprinting where methylation of 5′ regulatory regions can lead to transcriptional repression. Expression of a tumor suppressor gene can also be abolished by de novo DNA methylation of a normally unmethylated CpG.

Hypermethylation of genes encoding DNA repair enzymes can serve as markers for predicting the clinical response to certain cancer treatments. Certain chemotherapeutic agents (including alkylating agents for example) inhibit cellular proliferation by chemically modifying DNA, resulting in cell death. Treatment efforts with such agents can be thwarted and resistance to such agents develops because DNA repair enzymes repair the modified bases. In view of the deleterious side effects of most chemotherapeutic drugs, and the ineffectiveness of certain drugs for various treatments, it is desirable to predict the clinical response to treatment with chemotherapeutic agents.

U.S. Pat. No. 6,773,897 discloses methods relating to chemotherapeutic treatment of a cell proliferative disorder. In particular, a method is provided for “predicting the clinical response to certain types of chemotherapeutic agents”, including specific alkylating agents. The method entails determination and comparison of the methylation state of nucleic acid encoding a DNA repair enzyme from a patient in need of treatment with that of a subject not in need of treatment. Any difference is deemed “predictive” of response. The method, however, offers no suggestion of how to improve clinical outcome for any patient with an unfavorable “prediction”.

Temozolomide is an alkylating agent available from Schering Corp. under the trade name of Temodar® in the United States and Temodal® in Europe. Temodar® Capsules for oral administration contain temozolomide, an imidazotetrazine derivative. The chemical name of temozolomide is 3,4-dihydro-3-methyl-4-oxoimidazo[5,1-d]-as-tetrazine-8-carboxamide (see U.S. Pat. No. 5,260,291). The cytotoxicity of temozolomide or metabolite of it, MTIC, is thought to be primarily due to alkylation of DNA. Alkylation (methylation) occurs at the O⁶ position of guanine (5%), the N⁷ position of guanine (70%), and the N³ position of adenine (9%). O⁶-methylguanine is the primary cytotoxic lesion.

Temodar® (temozolomide) Capsules are currently indicated in the United States for the treatment of adult patients with newly diagnosed glioblastoma multiforme as well as refractory anaplastic astrocytoma, i.e., patients at first relapse who have experienced disease progression on a drug regimen containing a nitrosourea and procarbazine. Temodal® is currently approved in Europe for the treatment of patients with malignant glioma, such as glioblastoma multiforme or anaplastic astrocytoma showing recurrence or progression after standard therapy.

Although certain methods of treatment are effective for certain patients with proliferative diseases, there continues to be a great need for additional improved treatments. In view of the need for improved treatments for proliferative diseases, particularly cancers, novel methods of treatment would be a welcome contribution to the art. The present invention provides just such methods of treatment.

### SUMMARY OF THE INVENTION

The present invention provides methods for treating a patient with one or more cell proliferative disorders selected from the group consisting of melanoma, glioma, medulloblastoma, breast cancer, esophageal cancer, lung cancer, lymphoma, colorectal and/or colon cancer, head and neck cancer, and ovarian cancer, comprising administering to the patient a compressed temozolomide dosing schedule.

In one mode of this embodiment, one or more cell proliferative disorders is selected from the group consisting of melanoma, medulloblastoma, breast cancer, esophageal cancer, lung cancer, lymphoma, colorectal and/or colon cancer, head and neck cancer, and ovarian cancer, and the compressed temozolomide dosing schedule is as follows: 1000-2500 mg/m² administered within the first 1-5 days in a 14-day cycle; or 1000-2500 mg/m² administered within the first 1-5 days in a 28-day cycle. In select embodiments, the patient has not previously been treated with TMZ for glioblastoma multiforme or refractory anaplastic astrocytoma. In one such embodiment, the cell proliferative disorder is glioma. In another such embodiment, the cell proliferative disorder is melanoma.

One embodiment of the present invention methods for treating a patient with one or more cell proliferative disorders selected from the group consisting of melanoma, glioma, medulloblastoma, breast cancer, esophageal cancer, lung cancer, lymphoma, colorectal and/or colon cancer, head and neck cancer, and ovarian cancer, comprising administering to the patient a compressed temozolomide dosing schedule as follows: 1000-2500 mg/m² administered for 2 days in a 7-day or 8-day cycle; 1000-2500 mg/m² administered for 5 days in a 14-day or 15-day cycle; or 1000-2500 mg/m² administered for 10 days in a 28-day cycle; wherein the days over which the temozolomide dosing schedule is administered are intermittent.

As used herein, “treating” or “treatment” is intended to mean mitigating or alleviating a cell proliferative disorder in a mammal such as a human.

A cell proliferative disorder as described herein may be a neoplasm. Such neoplasms are either benign or malignant. The term “neoplasm” refers to a new, abnormal growth of cells or a growth of abnormal cells that reproduce faster than normal. A neoplasm creates an unstructured mass (a tumor) which can be either benign or malignant. The term “benign” refers to a tumor that is noncancerous, e.g., its cells do not invade surrounding tissues or metastasize to distant sites. The term “malignant” refers to a tumor that is cancerous, metastatic, invades contiguous tissue or is no longer under normal cellular growth control. In preferred embodiments, the methods and kits of the invention are used to treat cell proliferative disorders including but not limited to melanoma, glioma, medulloblastoma, prostate, esophageal cancer, lung cancer, breast cancer, ovarian cancer, testicular cancer, liver, kidney, spleen, bladder, colorectal and/or colon cancer, head and neck, carcinoma, sarcoma, lymphoma, leukemia or mycosis fungoides. In more preferred embodiments, the methods and kits of the invention are used to treat melanoma, glioma, medulloblastoma, esophageal cancer, lung cancer, lymphoma, colorectal and/or colon cancer, head and neck or ovarian cancer.

As used herein, the phrase “compressed dosing” with respect to TMZ refers to administering the same total dose of TMZ per treatment cycle over fewer days or over a reduced cycle time than previously prescribed (e.g., in Table 1). For example, administering the same total dose of TMZ per cycle, over fewer days than continuous daily dosing. Compressed dosing encompasses administering TMZ over a fewer number of days whether the days are intermittent or consecutive.

As used herein, the phrase “continuous daily dosing” with respect to TMZ refers to administering TMZ on a daily basis throughout a treatment cycle.

The present invention also provides kits for treating patients with cell proliferative disorders. The kits comprise: (1) reagents used in the methods of the invention; and (2) instructions to carry out the methods as described herein. The kits can further comprise temozolomide.

As would be understood by those skilled in the art, the novel methods and kits of the present invention for treating patients with cell proliferative disorders using temozolomide can be used as monotherapy or can be used in combination with radiotherapy and/or other cytotoxic and/or cytostatic agent(s) or hormonal agent(s) and/or other adjuvant therapy(ies).

### BRIEF DESCRIPTION OF THE DRAWINGS

FIG. 1 illustrates the number of DAOY human medulloblastoma cell colonies, a high MGMT level cell line, present after a 4-day cycle of TMZ treatment, where TMZ was administered according to one of two different dosing schedules: (i) continuous daily dosing (Day 1-4); or (ii) single pulse dosing (Day 1).

FIG. 2 illustrates the number of A375 human melanoma cell colonies, a high MGMT level cell line, present after a 4-day cycle of TMZ treatment, where TMZ was administered according to one of two different dosing schedules: (i) continuous daily dosing (Day 1-4); or (ii) single pulse dosing (Day 1). FIG. 3A illustrates the number of LOX human melanoma cell colonies, a low MGMT level cell line, present after a 4-day cycle of TMZ treatment, where TMZ was administered according to one of two different dosing schedules: (i) continuous daily dosing (Day 1-4); or (ii) single pulse dosing (Day 1).

FIG. 3B illustrates the number of LOX human melanoma cell colonies, a low MGMT level cell line, present after an 8-day cycle of TMZ treatment, where TMZ was administered according to one of three different dosing schedules: (i) continuous daily dosing (Day 1-8); (ii) dosing for 2 consecutive days (Day 1-2); or (ii) intermittent dosing for 2 days (Day 1, Day 5).

FIG. 4A illustrates the level of MGMT enzymatic activity in A375 human melanoma cells, a high MGMT level cell line, following TMZ treatment.

FIG. 4B illustrates the level of MGMT protein in A375 human melanoma cells, a high MGMT level cell line, following TMZ treatment. Lanes 1-4 reflect cell lysates prepared after 72 hours of TMZ treatment. Lanes 5-8 reflect cell lysates prepared after 72 hours of TMZ treatment followed by an additional 72 hours without TMZ treatment.

FIG. 5A illustrates the mean tumor growth curves of DAOY human medulloblastoma xenograft tumors, a high MGMT level cell line, following TMZ treatment for two consecutive 15-day cycles of continuous daily dosing (Day 1-15 (first cycle), Day 16-30 (second cycle)); where the total dose of TMZ administered was 0, 360, 540, or 810 mg per kg (mpk).

FIG. 5B illustrates the mean tumor growth curves of DAOY human medulloblastoma xenograft tumors, a high MGMT level cell line, following TMZ treatment for two consecutive 15-day cycles of dosing for 5 consecutive days (Day 1-5 (first cycle); Day 16-20 (second cycle)); where the total dose of TMZ administered was 0, 360, 540, or 810 mpk.

FIG. 5C illustrates mean tumor growth curves of DAOY human medulloblastoma xenograft tumors, a high MGMT level cell line, following TMZ treatment for two consecutive 15-day cycles of intermittent dosing for 5 days (Day 1, 4, 7, 10, 13 (first cycle); Day 16, 19, 22, 25, 28 (second cycle)); where the total dose of TMZ administered was 0, 360, 540, or 810 mpk.

FIG. 6 illustrates the individual tumor volume of A375 human melanoma xenograft tumors, a high MGMT level cell line, on Day 15 following a 15-day cycle of TMZ treatment, where TMZ was administered according to one of three different dosing schedules: (i) continuous daily dosing (Day 1-15); (ii) dosing for 5 consecutive days (Day 1-5); or (ii) intermittent dosing for 5 days (Day 1, 4, 7, 10, 13); where the total dose of TMZ administered was 0, 180, 270, or 405 mpk.

FIG. 7 illustrates the individual tumor volume of LOX human melanoma xenograft tumors, a low MGMT level cell line, on Day 18 following a 12-day cycle of TMZ treatment, where TMZ was administered according to one of two different dosing schedules: (i) continuous daily dosing (Day 1-12); or (ii) dosing for 4 consecutive days (Day 1-4); where the total dose of TMZ administered was 0, 36, 72, or 144 mpk.

FIG. 8 illustrates the mean tumor growth curves of U373 human glioma xenograft tumors, a high MGMT level cell line, following TMZ treatment for 5 consecutive days (Day 1-5) over a cycle that is at least a 28-day cycle; where the total dose of TMZ administered was 0, 175, or 350 mg per kg.

FIG. 9 illustrates the level of MGMT enzymatic activity in individual DAOY human medulloblastoma xenograft tumors, a high MGMT level cell line, following TMZ treatment for 5 consecutive days (where the total dose of TMZ administered was 0 or 405 mpk); as well as the level of MGMT enzymatic activity in untreated DAOY human medulloblastoma cells harvested from cell culture. C1, C2, and C3 represent tumors isolated from three different mice that had been treated with vehicle, while T1, T2, T3 represent tumors isolated from another three different mice that had been treated with TMZ.

### DETAILED DESCRIPTION OF THE INVENTION

The present invention provides novel methods and kits for treating a patient with a cell proliferative disorder, comprising administering to the patient a compressed temozolomide dosing schedule.

In one embodiment, the present invention provides a method for treating a patient with one or more cell proliferative disorders selected from the group consisting of melanoma, glioma, medulloblastoma, prostate, esophageal cancer, lung cancer, breast cancer, ovarian cancer, testicular cancer, liver, kidney, spleen, bladder, colorectal and/or colon cancer, head and neck, carcinoma, sarcoma, lymphoma, leukemia or mycosis fungoides, comprising administering to the patient a compressed temozolomide dosing schedule.

In one embodiment, the present invention provides a method for treating a patient with one or more cell proliferative disorders selected from the group consisting of melanoma, glioma, medulloblastoma, breast cancer, esophageal cancer, lung cancer, lymphoma, colorectal and/or colon cancer, head and neck cancer, and ovarian cancer, comprising administering to the patient a compressed temozolomide (TMZ) dosing schedule.

In one embodiment, one or more cell proliferative disorders is selected from the group consisting of melanoma, medulloblastoma, breast cancer, esophageal cancer, lung cancer, lymphoma, colorectal and/or colon cancer, head and neck cancer, and ovarian cancer, and the compressed temozolomide dosing schedule is as follows: 1000-2500 mg/m² administered within the first 1-5 days in a 14-day cycle; or 1000-2500 mg/m² administered within the first 1-5 days in a 28-day cycle.

In one embodiment, one or more cell proliferative disorders is melanoma and the compressed temozolomide dosing schedule is as follows: 1000-2500 mg/m² administered within the first 1-5 days in a 14-day cycle; or 1000-2500 mg/m² administered within the first 1-5 days in a 28-day cycle.

In one embodiment, one or more cell proliferative disorders is medulloblastoma and the compressed temozolomide dosing schedule is as follows: 1000-2500 mg/m² administered within the first 1-5 days in a 14-day cycle; or 1000-2500 mg/m² administered within the first 1-5 days in a 28-day cycle.

In one embodiment, one or more cell proliferative disorders is breast cancer and the compressed temozolomide dosing schedule is as follows: 1000-2500 mg/m² administered within the first 1-5 days in a 14-day cycle; or 1000-2500 mg/m² administered within the first 1-5 days in a 28-day cycle.

In one embodiment, one or more cell proliferative disorders is esophageal cancer and the compressed temozolomide dosing schedule is as follows: 1000-2500 mg/m² administered within the first 1-5 days in a 14-day cycle; or 1000-2500 mg/m² administered within the first 1-5 days in a 28-day cycle.

In one embodiment, one or more cell proliferative disorders is lung cancer and the compressed temozolomide dosing schedule is as follows: 1000-2500 mg/m² administered within the first 1-5 days in a 14-day cycle; or 1000-2500 mg/m² administered within the first 1-5 days in a 28-day cycle.

In one embodiment, one or more cell proliferative disorders is lymphoma and the compressed temozolomide dosing schedule is as follows: 1000-2500 mg/m² administered within the first 1-5 days in a 14-day cycle; or 1000-2500 mg/m² administered within the first 1-5 days in a 28-day cycle.

In one embodiment, one or more cell proliferative disorders is colorectal and/or colon cancer and the compressed temozolomide dosing schedule is as follows: 1000-2500 mg/m² administered within the first 1-5 days in a 14-day cycle; or 1000-2500 mg/m² administered within the first 1-5 days in a 28-day cycle.

In one embodiment, one or more cell proliferative disorders is head and neck cancer and the compressed temozolomide dosing schedule is as follows: 1000-2500 mg/m² administered within the first 1-5 days in a 14-day cycle; or 1000-2500 mg/m² administered within the first 1-5 days in a 28-day cycle.

In one embodiment, one or more cell proliferative disorders is ovarian cancer and the compressed temozolomide dosing schedule is as follows: 1000-2500 mg/m² administered within the first 1-5 days in a 14-day cycle; or 1000-2500 mg/m² administered within the first 1-5 days in a 28-day cycle.

In one embodiment, the compressed temozolomide dosing schedule is administered within the first 3-5 days in a 14-day cycle; or within the first 3-5 days in a 28-day cycle.

In one embodiment, one or more cell proliferative disorders is a glioma and the compressed temozolomide dosing schedule is as follows:
1000-2500 mg/m² administered within the first 1-5 days in a 14-day cycle;
1000-2500 mg/m² administered within the first 1-4 days in a 28-day cycle;
1001-2500 mg/m² administered within the first 1-5 days in a 28-day cycle; or
1000-2500 mg/m² administered within the first 1-5 days in a cycle greater than 28-days.

In one embodiment, the compressed temozolomide dosing schedule is as follows:
1000-2500 mg/m² administered within the first 3-5 days in a 14-day cycle;
1000-2500 mg/m² administered within the first 3-4 days in a 28-day cycle;
1001-2500 mg/m² administered within the first 3-5 days in a 28-day cycle; or
1000-2500 mg/m² administered within the first 3-5 days in a cycle greater than 28-days.

In one embodiment, one or more cell proliferative disorders is a glioma and the compressed temozolomide dosing schedule is as follows:
1000-2500 mg/m² administered within the first 1-5 days in a 28-day cycle wherein the patient has not previously been treated with temozolomide for glioblastoma multiforme or refractory anaplastic astrocytoma.

In one embodiment, the compressed temozolomide dosing schedule is administered within the first 3-5 days in a 28-day.

In one embodiment, the days over which the compressed temozolomide dosing schedule is administered are consecutive.

In one embodiment, the days over which the compressed temozolomide dosing schedule is administered are intermittent.

The present invention provides methods for treating a patient with one or more cell proliferative disorders selected from the group consisting of melanoma, glioma, medulloblastoma, breast cancer, esophageal cancer, lung cancer, lymphoma, colorectal and/or colon cancer, head and neck cancer, and ovarian cancer, comprising administering to the patient a compressed temozolomide dosing schedule as follows: 1000-2500 mg/m² administered for 2 days in a 7-day or 8-day cycle; 1000-2500 mg/m² administered for 5 days in a 14-day or 15-day cycle; or 1000-2500 mg/m² administered for 10 days in a 28-day cycle; wherein the days over which the temozolomide dosing schedule is administered are intermittent.

In one embodiment, one or more cell proliferative disorders is melanoma.

In one embodiment, one or more cell proliferative disorders is glioma.

In one embodiment, one or more cell proliferative disorders is medulloblastoma.

In one embodiment, one or more cell proliferative disorders is breast cancer.

In one embodiment, one or more cell proliferative disorders is esophageal cancer.

In one embodiment, one or more cell proliferative disorders is lung cancer.

In one embodiment, one or more cell proliferative disorders is lymphoma.

In one embodiment, one or more cell proliferative disorders is colorectal and/or colon cancer.

In one embodiment, one or more cell proliferative disorders is head and neck cancer.

In one embodiment, one or more cell proliferative disorders is ovarian cancer.

In one embodiment, the present invention provides kits comprising reagents and instructions for conducting the methods described above.

Also encompassed within the scope of the present invention are methods of administering temozolomide according to the methods taught herein in combination with a PARP inhibitor. The compelling evidence for the role of poly(ADP-ribose) polymerase(s) (PARP) in the cellular reaction to genotoxic stress was the stimulus to develop inhibitors as therapeutic agents to potentiate DNA-damaging anticancer therapies. Over the last two decades potent PARP inhibitors have been developed using structure activity relationships (SAR) and crystal structure analysis. These approaches have identified key desirable features for potent inhibitor-enzyme interactions. The resulting PARP inhibitors are up to 1,000 times more potent than the classical benzamides. These novel potent inhibitors have helped define the therapeutic potential of PARP inhibition. PARP inhibitors increase the antitumour activity of three classes of anticancer agents including temozolomide. A PARP inhibitor can be administered either prior to, concomitantly with or after administration of temozolomide as described herein. Exemplary PARP inhibitors include CEP-6800 (Cephalon; described in Miknyoczki et al., Mol Cancer Ther, 2(4):371-382 (2003)); 3-aminobenzamide (also known as 3-AB; Inotek; described in Liaudet et al., Br J Pharmacol, 133(8):1424-1430 (2001)); PJ34 (Inotek; described in Abdelkarim et al., Int J Mol Med, 7(3):255-260 (2001)); 5-iodo-6-amino-1,2-benzopyrone (also known as INH(2)BP; Inotek; described in Mabley et al., Br J Pharmacol, 133(6):909-919 (2001), GPI 15427 (described in Tentori et al., Int J Oncol, 26(2):415-422 (2005)); 1,5-dihydroxyisoquinoline (also known as DIQ; described in Walisser and Thies, Exp Cell Res, 251(2):401-413 (1999); 5-aminoisoquinolinone (also known as 5-AIQ; described in Di Paola et al., Eur J Pharmacol, 492 (2-3):203-210 (2004); AG 14361 (described in Bryant and Helleday, Biochem Soc Trans, 32(Pt 6):959-961 (2004); Veuger et al., Cancer Res, 63(18):6008-6015 (2003); and Veuger et al., Oncogene, 23(44):7322-7329 (2004)); ABT-472 (Abbott); INO-1001 (Inotek); AAI-028 (Novartis); KU-59436 (KuDOS; described in Farmer et al., “Targeting the DNA repair defect in BRCA mutant cells as a therapeutic strategy,” Nature, 434(7035):917-921 (2005)); and those described in Jagtap et al., Crit. Care Med, 30(5):1071-1082 (2002); Loh et al., Bioorg Med Chem Lett, 15(9):2235-2238 (2005); Ferraris et al., J Med Chem, 46(14):3138-3151 (2003); Ferraris et al., Bioorg Med Chem Lett, 13(15):2513-2518 (2003); Ferraris et al., Bioorg Med Chem, 11(17):3695-3707 (2003); Li and Zhang IDrugs, 4(7):804-812 (2001); Steinhagen et al., Bioorg Med Chem Lett, 12(21):3187-3190 (2002)); WO 02/06284 (Novartis); and WO 02/06247 (Bayer). In addition, a high-throughput screen for PARP-1 inhibitors is described in Dillon et al., J Biomol Screen, 8(3):347-352 (2003).

Also encompassed within the scope of the present invention are methods of administering temozolomide according to the methods taught herein in combination with a growth factor. According to a preferred embodiment, the growth factor is GM-CSF, G-CSF, IL-1, IL-3, IL-6, or erythropoietin. Non-limiting examples of growth factors include Epogen® (epoetin alfa), Procrit® (epoetin alfa), Neupogen® (filgrastim, a human G-CSF), Aranesp® (hyperglycosylated recombinant darbepoetin alfa), Neulasta® (also branded Neupopeg, pegylated recombinant filgrastim, pegfilgrastim), Albupoietin™ (a long-acting erythropoietin), and Albugranin™ (albumin G-CSF, a long-acting G-CSF). According to a more preferred embodiment, the growth factor is G-CSF.

As used herein, “GM-CSF” means a protein which (a) has an amino acid sequence that is substantially identical to the sequence of mature (i.e., lacking a signal peptide) human GM-CSF described by Lee et al., Proc. Natl. Acad. Sci. U.S.A., 82:4360 (1985) and (b) has biological activity that is common to native GM-CSF.

Substantial identity of amino acid sequences means that the sequences are identical or differ by one or more amino acid alterations (deletions, additions, substitutions) that do not substantially impair biological activity. Among the human GM-CSFs, nucleotide sequence and amino acid heterogeneity have been observed. For example, both threonine and isoleucine have been observed at position 100 of human GM-CSF with respect to the N-terminal position of the amino acid sequence. Also, Schrimsher et al., Biochem. J., 247:195 (1987), have disclosed a human GM-CSF variant in which the methionine residue at position 80 has been replaced by an isoleucine residue. GM-CSF of other species such as mice and gibbons (which contain only 3 methionines) and rats are also contemplated by this invention. Recombinant GM-CSFs produced in prokaryotic expression systems may also contain an additional N-terminal methionine residue, as is well known in the art. Any GM-CSF meeting the substantial identity requirement is included, whether glycosylated (i.e., from natural sources or from a eukaryotic expression system) or unglycosylated (i.e., from a prokaryotic expression system or chemical synthesis).

GM-CSF for use in this invention can be obtained from natural sources (U.S. Pat. No. 4,438,032; Gasson et al., supra; Burgess et al., supra; Sparrow et al., Wu et al., supra). GM-CSF having substantially the same amino acid sequence and the activity of naturally occurring GM-CSF may be employed in the present invention. Complementary DNAs (cDNAs) for GM-CSF have been cloned and sequenced by a number of laboratories, e.g., Gough et al., Nature, 309:763 (1984) (mouse); Lee et al., Proc. Natl. Acad. Sci. USA, 82:4360 (1985) (human); Wong et al., Science, 228:810 (1985) (human and gibbon); Cantrell et al., Proc. Natl. Acad. Sci. USA, 82:6250 (1985) (human), Gough et al., Nature, 309:763 (1984) (mouse); Wong et al., Science, 228:810 (1985) (human and gibbon); Cantrell et al., Proc. Natl. Acad. Sci. U.S.A., 82:6250 (1985) (human).

GM-CSF can also be obtained from Immunex, Inc. of Seattle, Wash. and Schering-Plough Corporation of Kenilworth, N.J. and from Genzyme Corporation of Boston, Mass.

In an advantageous embodiment of the present invention, temozolomide can be administered according to the methods taught herein in combination with an anti-emetic agent. Palonosetron, Tropisetron, Ondansetron, Granisetron, Bemesetron or a combination of at least two of the foregoing, very selective acting substances are employed as 5HT₃-receptor-antagonists which serve as enti-emetics. In this respect it is preferred that the amount of active anti-emetic substance in one dosage unit amounts to 2 to 10 mg, an amount of 5 to 8 mg active substance in one dosage unit being especially preferred. A daily dosage comprises generally an amount of active substance of 2 to 20 mg, particularly preferred is an amount of active substance of 5 to 16 mg. An NK-1 antagonist (neurokinin-1 antagonist) such as aprepitant alone or in combination with a steroid such as dexamethasone can also be used with or without a 5HT₃-receptor antagonist in the methods of the present invention. If necessary, those skilled in the art also know how to vary the active substance in a dosage unit or the level of the daily dosage according to the requirements. The factors determining this, such as body weight, overall constitution, response to the treatment and the like will constantly be monitored by the artisan in order to be able to react accordingly and adjust the amount of active substance in a dosage unit or to adjust the daily dosage if necessary.

According to yet another embodiment, temozolomide is administered using the methods taught herein in combination with a farnesyl protein transferase inhibitor.

According to other embodiments, temozolomide can be administered with another antineoplastic agent. Non-limiting examples of other useful antineoplastic agents include Uracil Mustard, Chlormethine, Cyclophosphamide, Ifosfamide, Melphalan, Chlorambucil, Pipobroman, Triethylenemelamine, Triethylenethiophosphoramine, Busulfan, Carmustine, Lomustine, Streptozocin, Dacarbazine, Methotrexate, 5-Fluorouracil, Floxuridine, Cytarabine, 6-Mercaptopurine, 6-Thioguanine, Fludarabine phosphate, Pentostatine, Gemcitabine, Vinblastine, Vincristine, Vindesine, Bleomycin, Dactinomycin, Daunorubicin, Doxorubicin, Epirubicin, Idarubicin, Paclitaxel, Mithramycin, Deoxycoformycin, Mitomycin-C, L-Asparaginase, Interferons, Etoposide, Teniposide 17.alpha.-Ethinylestradiol, Diethylstilbestrol, Testosterone, Prednisone, Fluoxymesterone, Dromostanolone propionate, Testolactone, Megestrolacetate, Tamoxifen, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, Goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, Oxaliplatin (Eloxatin®), Iressa (gefinitib, Zd1839), XELODA® (capecitabine), Tarceva® (erlotinib), Azacitidine (5-Azacytidine; 5-AzaC), and mixtures thereof.

Temozolomide may be administered with other anti-cancer agents such as the ones disclosed in U.S. Pat. Nos. 5,824,346, 5,939,098, 5,942,247, 6,096,757, 6,251,886, 6,316,462, 6,333,333, 6,346,524, and 6,703,400, all of which are incorporated by reference.

Non-limiting examples of dosing regimens and schedules are illustrated in Table 1.

TABLE 1
TMZ Dosing Regimens and Dose Intensity

| | | | | Dose/ | |
|---|---|---|---|---|---|
| Regimen | | | Total Dose | wk | Dose |
| No. | Dosing Regimen | Dosing schedule | (mg/m²/4 wks) | (mg/m²) | Intensity |
| 1 | 5/28 | 150-200 mg/m², 5 | 1000 | 250 | 1 |
| | | days/28 day cycle | | | |
| | | (200 mg) | | | |
| 2 | High doses 250 | 250 mg/m², 5/28, | 1250 | 312 | 1.2 |
| | mg/m² for 5/28 | concomitant w/a | | | |
| | | growth factor | | | |
| 3 | 14/28 | 100 mg/m², 14 | 1400 | 350 | 1.4 |
| | | days/28 day cycle | | | |
| 4 | High doses 300 | 300 mg/m², 5/28, | 1500 | 375 | 1.5 |
| | mg/m² for 5/28 | concomitant w/a | | | |
| | | growth factor | | | |
| 5 | 21/28 | 75 mg/m², 21 | 1575 | 393.75 | 1.6 |
| | | days/28 day cycle | | | |
| 6 | 42/56 | 75 mg/m², 6 wks/8 | 3150 | 393.75 | 1.6 |
| | | wk cycle | | | |
| 7 | 21/28 | 85 mg/m², 21 | 1785 | 446.25 | 1.8 |
| | | days/28 day cycle | | | |
| 8 | High doses 350 | 350 mg/m², 5/28, | 1750 | 437.5 | 1.8 |
| | mg/m² for 5/28 | concomitant w/a | | | |
| | | growth factor | | | |
| 9 | 14 on/7 off | 100 mg/m², 14 | 1400* | 467 | 1.9 |
| | | days/21 day cycle | | | |
| 10 | High doses 400 | 400 mg/m², 5/28, | 2000 | 500 | 2.0 |
| | mg/m² for 5/28 | concomitant w/a | | | |
| | | growth factor | | | |
| 11 | 7/7 | 150 mg/m², 7 | 2100 | 525 | 2.1 |
| | | days/14 day cycle | | | |
| 12 | 21/28 | 100 mg/m², 21 | 2100 | 525 | 2.1 |
| | | days/28 day cycle | | | |
| 13 | 14/28 | 150 mg/m², 14 | 2100 | 525 | 2.1 |
| | | days/28 day cycle | | | |
| 14 | Continuous | 75 mg/m², daily | 2100 | 525 | 2.1 |
| | dosing | | | | |
| 15 | High doses 450 | 450 mg/m², 5/28, | 2250 | 562.5 | 2.25 |
| | mg/m² for 5/28 | concomitant w/a | | | |
| | | growth factor | | | |
| 16 | 14 on/7 off | 150 mg/m², 14 | 2100* | 700 | 2.8 |
| | | days/21 day cycle | | | |
| 17 | Continuous | 100 mg/m², daily | 2800 | 700 | 2.8 |
| | dosing | | | | |
| 18 | High doses 250 | 250 mg/m², for 7/7, | 3500 | 875 | 3.5 |
| | mg/m² for 7/7 | concomitant with a | | | |
| | | growth factor | | | |
| 19 | High doses 300 | 300 mg/m², for 7/7, | 4200 | 1050 | 4.2 |
| | mg/m² for 7/7 | concomitant with a | | | |
| | | growth factor | | | |

*Represents total dose received in 3-week cycle

### EXPERIMENTS

A series of experimental studies were conducted as described below.

### Colony Formation Assays

As detailed below, DAOY human medulloblastoma cells (high MGMT level), A375 human melanoma cells (high MGMT level), and LOX human melanoma cells (low MGMT level) in in vitro colony formation assays were treated with different dosing schedules of TMZ. In brief, sub-confluent plates containing cells (DAOY, A375, or LOX) were trypsinized, then rinsed and suspended in appropriate culture medium before seeding in 6-well plates. Cells were incubated for 18-24 hours at 37° C. to allow cells to attach. Graded concentrations of TMZ or equivalent volumes of diluents were added in triplicate. Each pulse of TMZ lasted for 24 hours. For example, cells receiving continuous daily dosing of TMZ were treated with TMZ-containing medium every 24 hours throughout the cycle. Following the last pulse of TMZ in a cycle, TMZ-containing medium was removed and replaced with fresh medium without TMZ for the rest of the incubation period. Resulting colonies were stained with Crystal Violet solution and quantified using ImagePro plus software (Empire Imaging Systems, Inc. Asbury, N.J.).

### DAOY Human Medulloblastoma Cell Line (High MGMT Level)

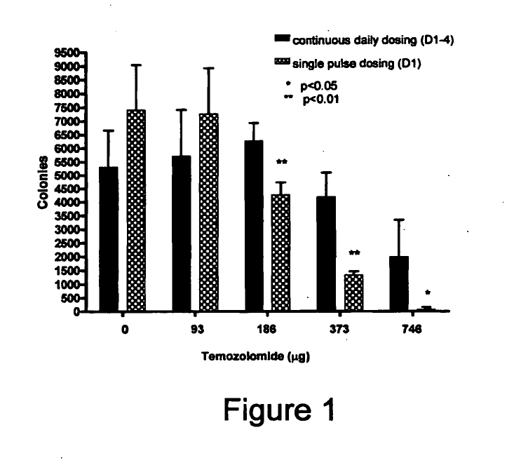

As illustrated in FIG. 1, colony formation assays were conducted whereby DAOY human medulloblastoma cells (high MGMT) were treated for a 4-day cycle according to one of two different TMZ dosing schedules: (i) continuous daily dosing (i.e., ¼ of total amount administered daily for four consecutive days; Day 1-4); or (ii) single pulse dosing (i.e., total amount administered in 1 day, Day 1); where the total amount of TMZ administered was 0, 93, 186, 373, or 746 μg. In short, single pulse dosing demonstrated better inhibition of colony formation than the continuous daily dosing at total TMZ levels of 186, 373, and 746 μg.

### A375 Human Melanoma Cell Line (High MGMT Level)

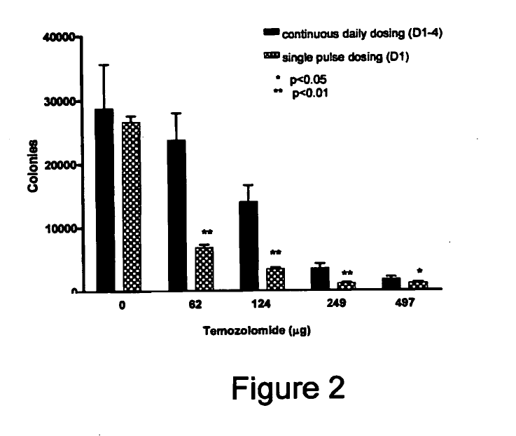

As illustrated in FIG. 2, colony formation assays were conducted whereby A375 human melanoma cells (high MGMT) were treated for a 4-day cycle according to one of two different TMZ dosing schedules: (i) continuous daily dosing (Day 1-4); or (ii) single pulse dosing (Day 1); where the total amount of TMZ administered was 0, 62, 124, 249, 497 μg. Interestingly, a similar pattern of response was observed in A375 human melanoma cells as that in DAOY human medulloblastoma cells. Dose-dependent inhibition by TMZ was demonstrated using both TMZ dosing schedules, but single pulse dosing resulted in better inhibition of colony formation than continuous daily dosing at total TMZ levels of 62, 124, 249, 497 μg.

### LOX Human Melanoma Cell Line (Low MGMT Level)

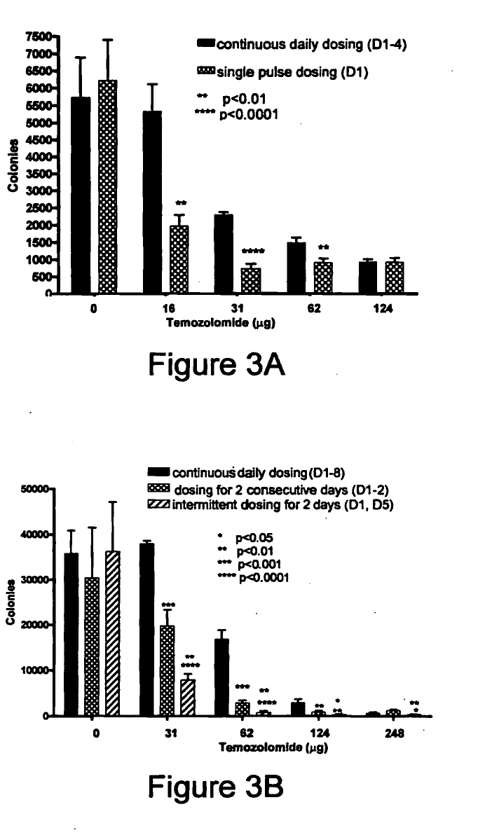

As illustrated in FIGS. 3A and 3B, colony formation assays were conducted whereby LOX human melanoma cells (low MGMT) were treated with TMZ dosing schedules for either a 4-day cycle (FIG. 3A) or an 8-day cycle (FIG. 3B).

In the 4-day cycle, illustrated in FIG. 3A, TMZ was administered according to one of two different dosing schedules: (i) continuous daily dosing (Day 1-4); or (ii) single pulse dosing (Day 1); where the total amount of TMZ administered was 0, 16, 31, 62, or 124 μg. Single pulse dosing demonstrated better inhibition of colony formation than continuous daily dosing.

In the 8-day cycle, illustrated in FIG. 3B, TMZ was administered according to one of three different dosing schedules: (i) continuous daily dosing (Day 1-8); (ii) dosing for 2 consecutive days (Day 1-2); or (ii) intermittent dosing for 2 days (Day 1, Day 5); where the total amount of TMZ administered was 0, 31, 62, 124, or 248 μg. Intermittent dosing for 2 days demonstrated better inhibition of colony formation than continuous daily dosing. In addition, intermittent dosing for 2 days demonstrated better inhibition of colony formation than dosing for 2 consecutive days at the same total TMZ dose.

### MGMT Assays

As detailed below, the enzymatic activity and protein level of MGMT were determined in A375 human melanoma cells following TMZ treatment at different total amounts 0, 58, 233, or 932 μg (corresponding to concentrations of 0, 10, 40, and 160 μM, respectively) for either: (i) 72 hours of TMZ treatment; or (ii) 72 hours of TMZ treatment followed by an additional 72 hours without TMZ treatment.

### MGMT Enzymatic Activity Assay

In brief, ³H-methylated DNA substrate was prepared from calf thymus DNA. This substrate was incubated with 50 μg of cell extract at 37° C. for 45 min. After a complete transfer of radioactivity to MGMT protein, excess DNA was hydrolyzed and washed with trichloroacetic acid (TCA). Radioactivity transferred to MGMT protein was measured by scintillation counting.

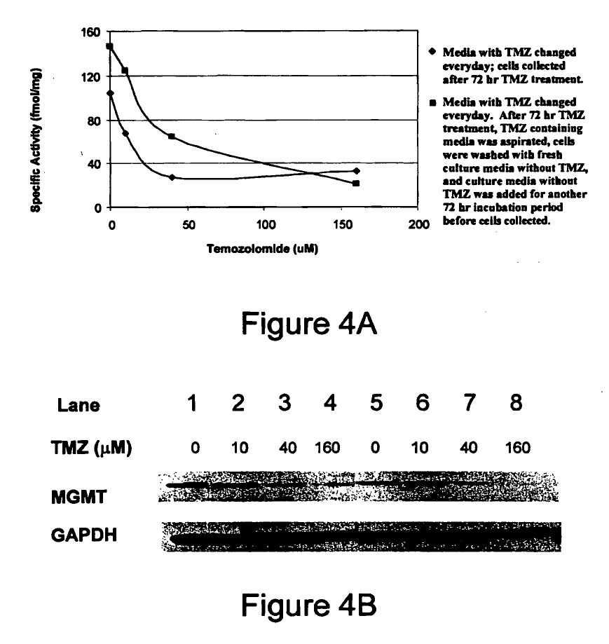

As illustrated in FIG. 4A, the level of MGMT enzymatic activity was measured in A375 melanoma cells following TMZ treatment at different total amounts 0, 58, 233, or 932 μg (corresponding to concentrations of 0, 10, 40, and 160 μM, respectively). Treatment of TMZ for 72 hours caused dose-dependent reduction of MGMT. Moreover, to evaluate how long the reduction of MGMT activity persists after removal of drug treatment, enzyme activity was also measured in a parallel set of cells that, after the 72-hour treatment, were washed and maintained in medium without TMZ for another 72 hours. Interestingly, the enzyme activity remained reduced in a dose-dependent manner for 72 hours after drug removal. This indicates that high dose pulse treatment of TMZ has a prolonged effect on the level of MGMT, which also indicates that a subsequent dose of TMZ treatment of these cells may potentiate the cytotoxicity of TMZ.

### MGMT Western Blot

Tumor cells (5×10⁵) were seeded in 100 mm×20 mm culture plates containing 10 ml of 90% DMEM (GIBCO, N.Y.) with 10% fetal bovine serum.

Cells were treated with increasing concentrations of TMZ or equivalent volume of diluents. At various times after treatment, whole-cell lysates were prepared in a solution containing 10 mM Tris-HCl (pH7.5), 10 mM NaH₂PO₄/NaHPO₄, 130 mM NaCl, 1% Triton X-100, 10 mM PPi (BD Biosciences Pharmingen). Equal amounts of total protein were electrophoresed on a 4-12% SDS-polyacrylamide gel and electrotransferred to polyvinylidene difluoride membranes. The blots were blocked with 5% non-fat dry milk in Tris buffered saline (TBS) and probed with specific antibodies against MGMT (BD Bioscience Pharmingen) or against GAPDH (USBiological) as an internal control.

As illustrated in FIG. 4B, the level of MGMT protein was assayed by Western blot in A375 melanoma cells following TMZ treatment at different total amounts 0, 58, 233, or 932 μg (corresponding to concentrations of 0, 10, 40, and 160 μM, respectively). Lanes 1-4 reflect cell lysates prepared after 72 hours of TMZ treatment. Lanes 5-8 reflect cell lysates prepared after 72 hours of TMZ treatment followed by an additional 72 hours without TMZ treatment. The level of MGMT protein level detected correlated to the level of MGMT specific activity measured in similarly treated cells described in FIG. 4A. In both assays, a dose-dependent reduction in MGMT protein level was detected.

### In Vivo Studies

As detailed below, different TMZ dosing schedules were evaluated in xenograft tumors formed using DAOY human medulloblastoma cells (high MGMT level), U373 human glioma cells (high MGMT level), A375 human melanoma cells (high MGMT level), and LOX human melanoma cells (low MGMT level).

In brief, female athymic nude mice or female SCID mice (4-6 week old) from Charles River Laboratories were maintained in a VAF-barrier facility. Animal procedures were performed in accordance with the rules set forth in the N.I.H. guide for the care and use of laboratory animals.

DAOY human medulloblastoma cells (5×10⁶), U373 human glioma cells (5×10⁶), LOX human melanoma cells (5×10⁵), and A375 human melanoma cells (5×10⁶) were inoculated subcutaneously in the right flank of the animal (LOX in SCID mice; DAOY, U373, and A375 in nude mice). To facilitate in vivo growth, Matrigel was mixed with DAOY and A375 cells (50%) before inoculation. When tumor volumes were approximately 100 mm³, animals were randomized and grouped (n=10 LOX, DAOY, and A375; n=9 U373). Tumor volumes and body weight were measured twice weekly using Labcat™ computer application (Innovative Programming Associates, N.J.). Tumor volumes were calculated by the formula (W×L×H)×π×⅙. TMZ was administered by intraperitoneal injections with 20% HPβCD (containing 1% DMSO) as vehicle.

Mice bearing xenograft tumors of DAOY human medulloblastoma cells, a high MGMT level cell line, were treated with one of three different dosing schedules. In a 15-day cycle, under same total dose levels, mice received one of the following TMZ treatments: (i) day 1 through day 15; (ii) day 1 through day 5; or (iii) intermittently on day 1, 4, 7, 10, and 13. For all dosing schedules, three different dose levels (180, 270, and 405 mg/kg total) were used.

Mice bearing xenograft tumors of U373 human glioma cells, a high MGMT level cell line, were treated with TMZ for 5 consecutive days (day 1 through day 5) over a cycle that is at least a 28-day cycle. TMZ was administered by intraperitoneal injection at a dose level of either: (i) 35 mg/kg/day or (ii) 70 mg/kg/day; resulting in a cumulative total dose level of 175 or 350 mg/kg, respectively.

Mice bearing xenograft tumors of A375 human melanoma cells, a high MGMT level cell line, were treated with three different dosing schedules. Similar to the schedules used for the DAOY model, in a 15-day cycle, under same total dose levels, mice received one of the following TMZ treatments: (i) day 1 through day 15; (ii) day 1 through day 5; or (iii) intermittently on day 1, 4, 7, 10, and 13. For all dosing schedules, three different dose levels (180, 270, and 405 mg/kg total) were used.

Mice bearing xenograft tumors of LOX human melanoma cells, a low MGMT level cell line, were treated with TMZ using two different schedules. The same total dose was administered evenly divided over the course of either: (i) 4 or (ii) 12 days. TMZ was administered through intraperitoneal injection using cumulative total dose levels of 36, 72 or 144 mg/kg.

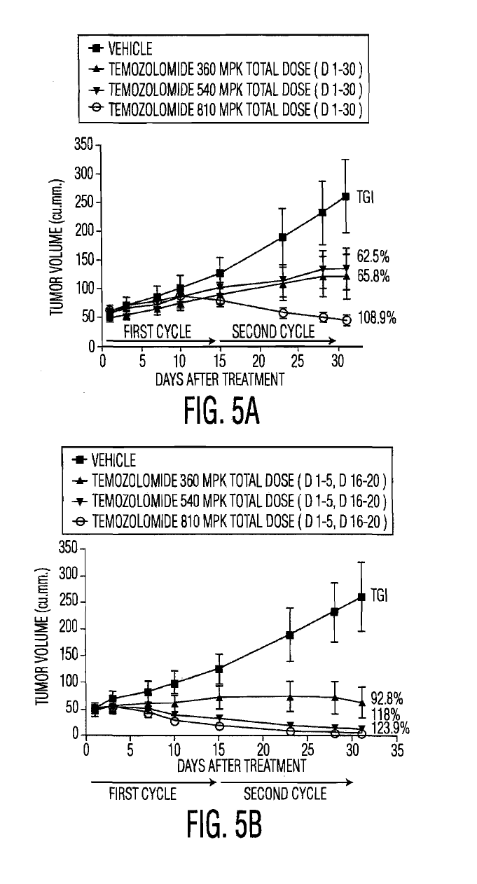

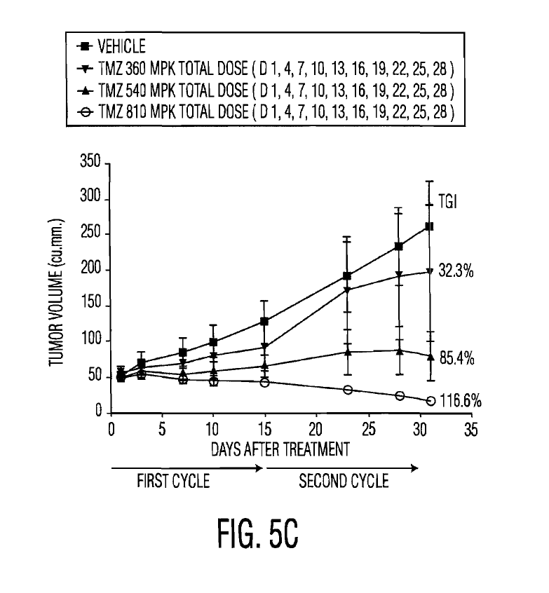

As illustrated in FIG. 5, nude mice bearing xenograft tumors of DAOY human medulloblastoma cells, a high MGMT level cell line, were treated with three different schedules of TMZ in a dose-dependent fashion. FIG. 5A illustrates the mean tumor growth curves of DAOY human medulloblastoma xenograft tumors following TMZ treatment for two consecutive 15-day cycles of continuous daily dosing (Day 1-15 (first cycle), Day 16-30 (second cycle)); where the total dose of TMZ administered was 0, 360, 540, or 810 mg per kg (mpk). FIG. 5B illustrates the mean tumor growth curves of DAOY human medulloblastoma xenograft tumors following TMZ treatment for two consecutive 15-day cycles of dosing for 5 consecutive days (Day 1-5 (first cycle); Day 16-20 (second cycle)); where the total dose of TMZ administered was 0, 360, 540, or 810 mpk. FIG. 5C illustrates mean tumor growth curves of DAOY human medulloblastoma xenograft tumors following TMZ treatment for two consecutive 15-day cycles of intermittent dosing for 5 days (Day 1, 4, 7, 10, 13 (first cycle); Day 16, 19, 22, 25, 28 (second cycle)); where the total dose of TMZ administered was 0, 360, 540, or 810 mpk. Notably, the mean tumor volume of each treatment group during the period of therapy is represented. In this tumor model, both the dosing for 5 consecutive days and the intermittent dosing for five days demonstrated better tumor growth inhibition than the continuous daily dosing schedule (Day 1-15). In fact, tumor regression occurred after merely one cycle of treatment with either the two higher dose levels of TMZ (54 or 81 mg/kg/day) in the dosing for 5 consecutive days as well as with the highest dose level of TMZ (81 mg/kg/day) in the intermittent dosing schedule.

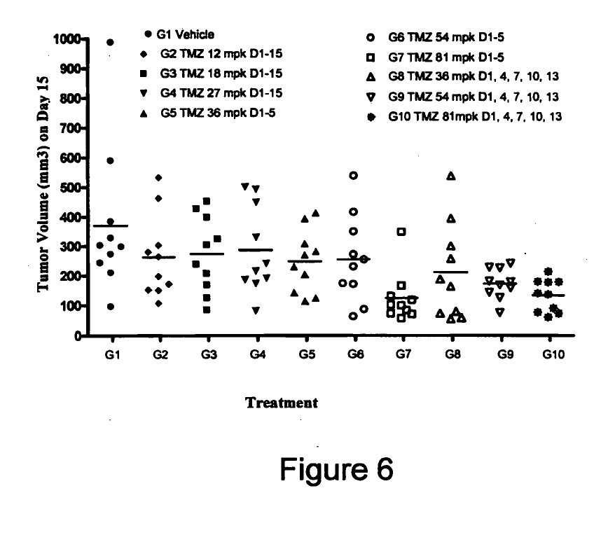

As illustrated in FIG. 6, nude mice bearing xenograft tumors of A375 human melanoma cells, a high MGMT cell line, were treated with the same dosing schedules as were mice in the DAOY human medulloblastoma xenograft tumor study discussed above. A similar pattern was observed in A375 human melanoma xenograft tumors as those of DAOY medulloblastoma xenograft tumors. Notably, the two higher dose levels of intermittent dosing schedule (Day 1, 4, 7, 10, 13) and the highest dose level of the dosing for 5 consecutive days (Day 1-5) generated significantly better efficacy than the equivalent dose levels of the continuous daily dosing schedule (Day 1-15).

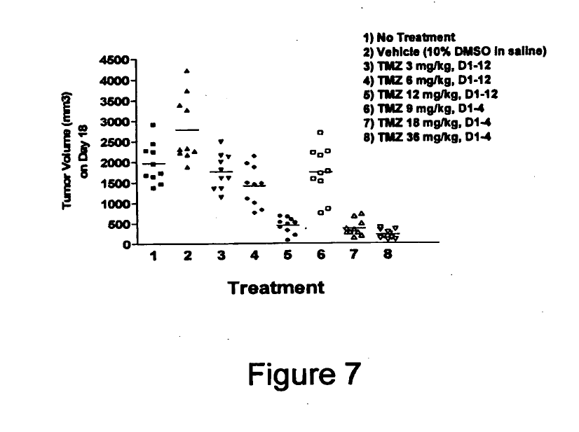

As illustrated in FIG. 7, SCID mice bearing xenograft tumors of LOX melanoma cells, a low MGMT cell line, were treated with two different dosing schedules for a 12-day cycle: (i) dosing for 4 consecutive days (Day 1-4); or (ii) continuous daily dosing (Day 1-12). At the intermediate dose (72 mg/kg), the 4-day treatment schedule induced significantly better efficacy (88% TGI) than the 12-day schedule (50% TGI). In contrast, no statistical difference was observed at higher and lower dose levels. The efficacy of TMZ was schedule dependent, with greater efficacy seen when dosing for 4 consecutive days.

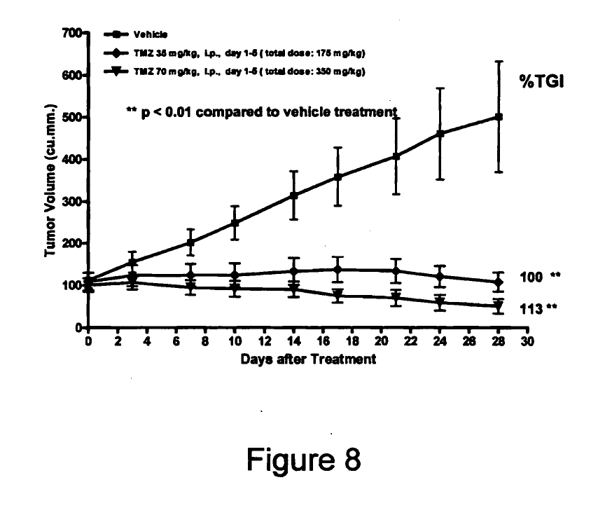

As illustrated in FIG. 8, nude mice bearing xenograft tumors of U373 human glioma cells, a high MGMT cell line, were treated with TMZ for 5 consecutive days (Day 1-5) over a cycle that is at least a 28-day cycle at a dose level of either: (i) 35 mg/kg or (ii) 70 mg/kg resulting in a cumulative total dose level of 175 or 350 mg/kg, respectively. The efficacy of TMZ was dose dependent, with better efficacy (113% TGI) at the 70 mg/kg dose level as compared to (100% TGI; i.e., stasis) at the 35 mg/kg dose level. Notably, the tumor growth inhibition in both TMZ treatments is significant (p<0.01) as examined by Student's t test (unpaired, 2 tailed).

### Intratumoral MGMT Enzymatic Activity

In brief, three DAOY tumors treated with either 81 mg/kg TMZ or vehicle for five consecutive days were collected from mice. Each tumor was homogenized and processed for MGMT enzymatic activity following treatment. MGMT activity measured from untreated DAOY cells was also included as a control.

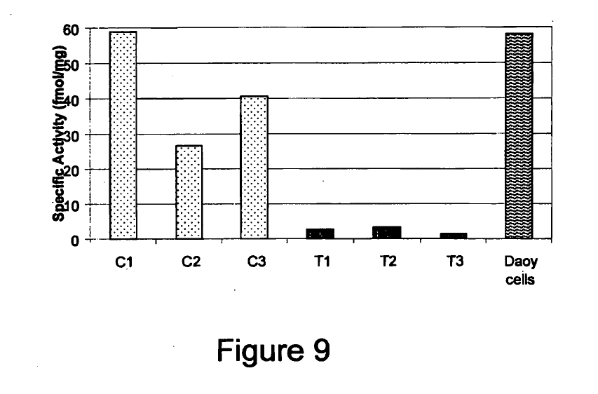

As illustrated in FIG. 9, unlike tumors treated with vehicle which had similar level of MGMT activity compared to DAOY cells harvested from cell culture, tumors that had been treated for five consecutive days with TMZ had little MGMT activity detected.

### SUMMARY

These studies demonstrate that compressed dosing schedules of TMZ are more efficacious than continuous daily dosing schedules of TMZ at inhibiting cell growth as demonstrated in in vitro colony formation assays and in vivo in xenograft models.

Discussion or citation of a reference herein shall not be construed as an admission that such reference is prior art to the present invention.

Although certain presently preferred embodiments of the invention have been described herein, it will be apparent to those skilled in the art to which the invention pertains that variations and modifications of the described embodiments may be made without departing from the spirit and scope of the invention. Accordingly, it is intended that the invention be limited only to the extent required by the appended claims.

We claim:

1. A method for treating a patient with one or more cell proliferative disorders selected from the group consisting of melanoma, glioma, medulloblastoma, breast cancer, esophageal cancer, lung cancer, lymphoma, colorectal and/or colon cancer, head and neck cancer, and ovarian cancer, comprising administering to the patient a compressed temozolomide dosing schedule.

2. The method of claim 1, wherein one or more cell proliferative disorders is selected from the group consisting of melanoma, medulloblastoma, breast cancer, esophageal cancer, lung cancer, lymphoma, colorectal and/or colon cancer, head and neck cancer, and ovarian cancer, and the compressed temozolomide dosing schedule is as follows:
1000-2500 mg/m² administered within the first 1-5 days in a 14-day cycle; or
1000-2500 mg/m² administered within the first 1-5 days in a 28-day cycle.

3. The method of claim 2, wherein the compressed temozolomide dosing schedule is administered within the first 3-5 days in a 14-day cycle; or within the first 3-5 days in a 28-day cycle.

4. The method of claim 1, wherein one or more cell proliferative disorders is a glioma and the compressed temozolomide dosing schedule is as follows:
1000-2500 mg/m² administered within the first 1-5 days in a 14-day cycle;
1000-2500 mg/m² administered within the first 1-4 days in a 28-day cycle;
1001-2500 mg/m² administered within the first 1-5 days in a 28-day cycle; or
1000-2500 mg/m² administered within the first 1-5 days in a cycle greater than 28-days.

5. The method of claim 4, wherein the compressed temozolomide dosing schedule is as follows:
1000-2500 mg/m² administered within the first 3-5 days in a 14-day cycle;
1000-2500 mg/m² administered within the first 3-4 days in a 28-day cycle;
1001-2500 mg/m² administered within the first 3-5 days in a 28-day cycle; or
1000-2500 mg/m² administered within the first 3-5 days in a cycle greater than 28-days.

6. The method of claim 1, wherein one or more cell proliferative disorders is a glioma and the compressed temozolomide dosing schedule is as follows:
1000-2500 mg/m² administered within the first 1-5 days in a 28-day cycle wherein the patient has not previously been treated with temozolomide for glioblastoma multiforme or refractory anaplastic astrocytoma.

7. The method of claim 6, wherein the compressed temozolomide dosing schedule is administered within the first 3-5 days in a 28-day.

8. The method of any one of claims 1-7, wherein the days over which the compressed temozolomide dosing schedule is administered are consecutive.

9. The method of any one of claims 1-7, wherein the days over which the compressed temozolomide dosing schedule is administered are intermittent.

10. A method for treating a patient with one or more cell proliferative disorders selected from the group consisting of melanoma, glioma, medulloblastoma, breast cancer, esophageal cancer, lung cancer, lymphoma, colorectal and/or colon cancer, head and neck cancer, and ovarian cancer, comprising administering to the patient a compressed temozolomide dosing schedule as follows:
1000-2500 mg/m² administered for 2 days in a 7-day or 8-day cycle;
1000-2500 mg/m² administered for 5 days in a 14-day or 15-day cycle; or
1000-2500 mg/m² administered for 10 days in a 28-day cycle;
wherein the days over which the temozolomide dosing schedule is administered are intermittent.

11. A kit comprising reagents and instructions for conducting the method according to claim 10.
